(19) United States
(12) Patent Application Publication
(10) Pub. No.: US 2007/0039619 A1
(43) Pub. Date: Feb. 22, 2007

(54) Manual resuscitator with oxygen tubing reservoir

(76) Inventors: Ole B. Kohnke, Fredericksberg (DE); Jakob Bonnelykke Kristensen, Olstykke (DE)

Correspondence Address: Paul Grandinetti;Levy & Grandinetti, Suite 408, 1725 K Street, N.W., Washington, DC, 20006-1419, US

(21) Appl. No.: 11/207,790

(22) Filed: Aug. 22, 2005

(51) Int. Cl. A61M 16/00

(52) U.S. Cl. 128/205.13; 128/205.24

(57) ABSTRACT

A manual resuscitator is disclosed which includes a bag, a bag inlet valve assembly, and an open oxygen reservoir tube, a conduit between the bag inlet valve assembly and the open oxygen reservoir tube, and a one-way relief valve connecting the conduit to the atmosphere. The one-way relief valve has an opening pressure sufficiently low to prevent excitation of resonance pressure vibrations in the open oxygen reservoir tube.

### BACKGROUND OF THE INVENTION

1. Field of the Invention

The present invention relates to manual resuscitators. More particularly, the present invention relates to manual resuscitators comprising oxygen tubing reservoir attachments that are resistant to vibration and premature closing.

2. Description of Related Art

Manual resuscitators having oxygen tubing reservoir attachments are well known and have been described in several patents. Such devices serve to provide artificial ventilation with mixtures of room air and supplementary oxygen and generally include the following basic components: a squeezable, self-expanding bag; a directional valve that controls the ventilatory flow to and from the patient; a one way inlet valve that allows the bag to draw in a mixture of air and oxygen during expansion; an elongated tubing reservoir sealingly attached to the inlet valve housing at one end and open to the atmosphere at the other end; and an oxygen supply inlet located upstream, adjacent to the bag inlet valve, for the supply of a continuous flow of supplementary oxygen from an oxygen flow meter into the tubing reservoir. These devices, however, do not include means for preventing vibrations as gases flow through the components and do not include means for preventing premature closing of the passageways for exhalation.

The bag is generally made from an elastomeric material and is designed to expand and refill quickly following the squeezing of its content to the patient. The directional control valve has one port connected to the interior of the bag, a second port connected to the patient's airway via a face mask or an airway tube, and a third port leading to the atmosphere. Control members in the valve housing (1) direct the flow of the breathing mixture from the first port to the second port and close the third port when the bag is squeezed and (2) direct the flow of the exhalation breathing mixture from the second port to the third port and close the first port when the bag is released for expansion and refilling.

The switching of the valve is controlled by pressure differences created in the three ports during squeezing and releasing of the bag. The one-way inlet valve for refilling the bag with air and oxygen mixture is controlled by the pressure difference between the bag and the pressure inside an oxygen reservoir tubing connection. The oxygen reservoir normally comprises lightweight, corrugated tubing wherein the dimensions of the cross section of the tubing are selected to obtain a backwards flow front of supplementary oxygen over the entire cross section when the bag is squeezed or at rest in the fully expanded position. The oxygen flow backwards thereby pushes the air inside the reservoir tubing backwards with a minimum of turbulent mixing at the flow front and out through the open end to the atmosphere. The result of this is that the volume of oxygen accumulated in the reservoir will be sucked into the bag first when the bag expands and will refill the ventilation volume squeezed out.

If the volume of the reservoir tube is equal to or larger than the ventilation volume, the concentration of oxygen in the breathing mixture may be adjusted to any desired concentration between room air (21% oxygen) and pure oxygen by adjusting the flow of supplemental oxygen. The oxygen supply tubing is either connected to an inlet nipple at the bag inlet valve housing or is placed inside the reservoir tubing and out through the open end.

U.S. Pat. No. 3,366,133 discloses a breathing device for preventing loss and providing uniform flow of a supplementary medium. A supply tube is connected at one end to a valve for controlling the flow of gas to and from a point of use and at its other end to a self-expanding bag. Intermediate its ends the supply tube is connected to a branch tube for introduction of a supply medium at the end thereof remote from the supply tube. An elongated path reservoir is in communication at one of its ends to the branch tube in the vicinity of the connection of the latter with the supply medium, and the reservoir is in communication at its other end with atmosphere.

U.S. Pat. No. 5,279,289 discloses a resuscitator regulator that is provided with an integral carbon dioxide detector for indicating the presence of carbon dioxide in a patient. The carbon dioxide detector is a conventional pH sensitive chromogenic compound that is positioned in the breathing circuit to permit a rapid indication. Additionally, a plug is provided for sealing the indicator from the atmosphere while in storage to prevent contamination and degradation of the indicator compound.

Canadian Patent Number 1,220,111 discloses a resuscitation apparatus for use during medical procedures. The device comprises a squeeze bag having a gas inlet and a gas outlet, and a specifically configured valve joined to the bag over the gas outlet. The valve housing includes a squeeze bag port in flow communication with the gas outlet opening, a patient port and an exhalation port. The valve disposed in the housing includes a portion for directing fluid from the squeeze bag through the patient port during inhalation or forced respiration and through the exhalation port during exhalation. Another portion of the valve closes the exhalation port during inhalation or forced respiration such that fluid from the squeeze bag is directed to the patient.

Several commercially available resuscitators are designed as described above, but most of them do not function in the manner desired towards the end of the patient's expiration of breath. These devices tend to cause the control members in the directional control valve to start vibrating and eventually close to the atmosphere prematurely during expiration of breath, leaving an unintended positive pressure in the lungs of the patient. This condition is not desirable, and the industry lacks a manual resuscitator that:
(1) is simple and inexpensive to operate and manufacture,
(2) includes means for preventing vibrations as gases flow through the components, and
(3) includes means for preventing premature closing of the passageways for exhalation.

It is an object of the present invention to provide a resuscitator with means to prevent such vibrations and premature closing, while retaining the tubing reservoir concept preferred by many users over other oxygen accumulation systems.

### SUMMARY OF THE INVENTION

The present invention includes a manual resuscitator having a bag, a bag inlet valve assembly, and an open oxygen reservoir tube. There is a conduit between the bag inlet valve assembly and the open oxygen reservoir tube, and a one-way relief valve connecting the conduit to the atmosphere. The one-way relief valve has an opening pressure sufficiently low to prevent excitation of resonance pressure vibrations in the open oxygen reservoir tube.

In a preferred embodiment, the one-way relief valve includes an elastomeric valve diaphragm having an outer edge resting against a valve seat and a central portion attached to recessed means of the valve. The elastic deformation of the diaphragm pre-strains the outer edge of the diaphragm against the valve seat and thereby defines the opening pressure of the valve.

More preferably, the one-way relief valve has an opening pressure sufficient to substantially prevent leakage of supplementary oxygen through the valve to the atmosphere during backwards flow in the oxygen reservoir.

### BRIEF DESCRIPTION OF THE DRAWINGS

FIG. 1 illustrates a sectional side view of the manual resuscitator of the invention in the fully expanded state of the bag.

FIG. 2 illustrates a sectional side view of the manual resuscitator of the invention during squeezing of the bag.

FIG. 3 illustrates a sectional side view of the manual resuscitator of the invention during expansion and refilling of the bag.

FIG. 4 illustrates a sectional side view of the manual resuscitator of the present invention at the exact moment when the wall of the bag has unfolded from concave to convex curvature.

### DETAILED DESCRIPTION OF THE INVENTION

The present invention includes a manual resuscitator having a bag and a bag inlet valve assembly. The bag inlet valve assembly is connected to an open oxygen reservoir tube. There is a conduit between the bag inlet valve assembly and the open oxygen reservoir tube, the conduit having a one-way relief valve that connects the conduit to the atmosphere. The one-way relief valve has an opening pressure sufficiently low to prevent excitation of resonance pressure vibrations in the open oxygen reservoir tube.

The manual resuscitator of the invention includes an element known simply in the art as a “bag.” The bag of the manual resuscitator is a body or container that can hold a volume of air, oxygen, other gases, and combinations of gases. The bag can be of any desired shape and size, but is typically of a shape and size that can be held in a person's hand and operated by one hand. The bag provides a cavity containing the air or other gases for inhalation. The volume of the bag is, desirably, sufficient to ventilate the intended child or adult patient.

Bags are usually made from impermeable materials that are flexible and, if the material itself is non-resilient, can also include a material that is resilient to compression. A resiliently flexible material can include a polymer, elastomer, natural or synthetic rubber, or other flexible web material such as paper. Desirably, the material permits the bag to be compressed and then returned to its original shape. Bags made of polymer, elastomer, and rubber materials, for example, have such properties. If a flexible web material, such as paper is used, a spring or other resilient means can be employed to return the flexible web material to its original shape and condition after the bag is compressed.

The present invention also includes a bag inlet valve assembly. The bag inlet valve assembly is connected to a port or opening in the bag. The bag inlet valve assembly can vary in complexity, as well as in the number of its components or elements, and is a means for transferring or feeding gas unidirectionally into the bag.

A means for transferring gas includes at least one vent or passageway to at least a first source of gas. Desirably, the first source of gas is atmospheric air or “an open oxygen reservoir tube,” but can be another gas or gas mixture. Desirably, the means for transferring gas includes a conduit within which different gases from at least two sources can be mixed before entering the bag. The means for transferring gas permits unidirectional flow of the gas or mixed gases through a regulator means or valve.

Regulating means opens as gas passes into the bag and closes to prevent gas from exiting the bag and can be operated by tensioning means, such as a spring, a resilient metal, or a polymer plate. Alternatively, the regulating means can, itself, be made from a resilient material to independently provide this function of opening and closing to control the unidirectional flow of gas. The regulating means can also be operated by pressure differentiation between its bag side and its inlet valve assembly side. The regulating means can include a valve to control the unidirectional flow of gas from the inlet valve assembly into the bag and can optionally include a second source of gas, such as a feed line to oxygen.

The present invention includes a directional control valve assembly connected to a port or opening in the bag. The control valve assembly can vary in complexity as well as in the number of its components or elements. The directional control valve is a means for ventilating a patient and supplies gas unidirectionally from the bag to a patient and from the patient to the atmosphere.

The directional control valve assembly, desirably, has a gas pressure sensitive member for controlling the direction of gas flow to and from the patient, which can be an elastomeric control member. The pressure sensitive member permits gas to flow from the bag to a first gas exit port, the “patient port,” when the pressure inside the bag is higher than the pressure in the patient port. When the pressure in the patient port is higher than the pressure inside the bag, the pressure sensitive member permits gas to flow from the patient port via a second gas exit port, the “exhaust port,” to atmosphere. In case the pressure of the gas being fed to the directional control valve assembly is vibrating, the pressure sensitive member can be excited into vibrations between its two positions, causing increased resistance to exhalation and premature closing of the passageway to atmosphere. To prevent such pressure vibrations from being created by excitation of resonance pressure vibrations in the oxygen reservoir tube, a one-way pressure relief valve is provided between the conduit connecting the bag inlet valve assembly and the open oxygen reservoir tube. The relief valve connects the conduit to atmosphere and has an opening pressure sufficiently low to prevent excitation of resonance vibrations in the tube.

In a preferred embodiment, the one-way relief valve comprises an elastomeric valve diaphragm having an outer edge resting against a valve seat and a central portion attached to recessed means of the valve. The elastic deformation of the diaphragm pre-strains the outer edge of the diaphragm against the valve seat. More preferably, the one-way relief valve has an opening pressure sufficient to substantially prevent leakage of supplementary oxygen through the valve to the atmosphere during backwards flow in the oxygen reservoir.

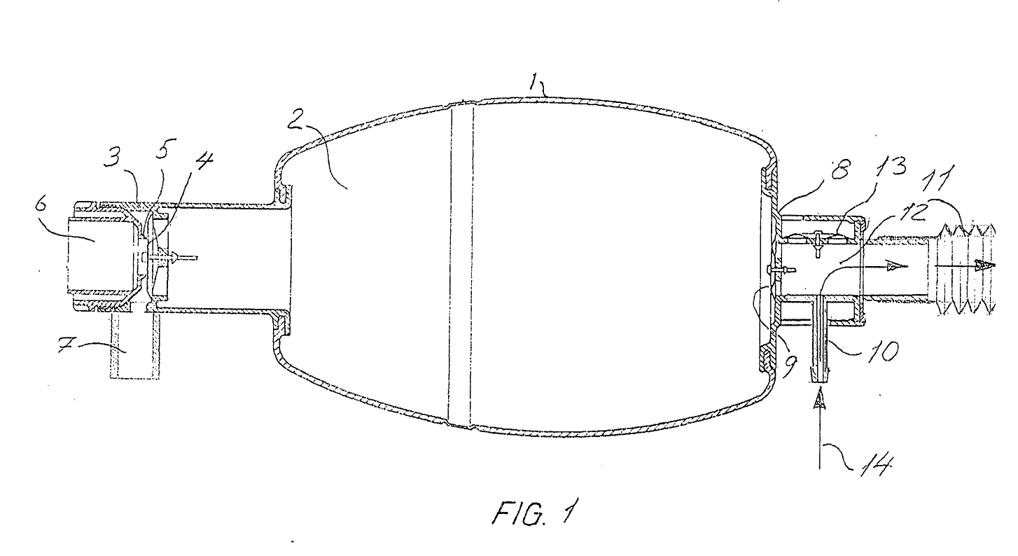

FIG. 1 illustrates, as a currently preferred embodiment, a sectional side view of the manual resuscitator of the present invention in the fully expanded state of a bag 1 having two oppositely disposed ends. A reservoir 2 inside the bag 1 contains a volume of a breathing mixture of air or other gas. A directional control valve 5 is attached to one end of a bag outlet assembly 3 of the bag 1. A bag inlet assembly 8 is attached to the opposite end of the bag 1 and a cut-off section of a corrugated tubing oxygen reservoir 11 is attached to an inlet valve conduit 12. An oxygen supply inlet nipple 10 opens into the inlet valve conduit 12 and supplementary oxygen flow 14 pushes air in the oxygen reservoir 11 backwards as indicated with arrows. A pre-strained pressure relief valve 13 closes side holes located in the sidewall of the inlet valve conduit 12 and prevents the inflow of oxygen from leaking therethrough to the atmosphere.

Alternatively, the directional control valve, the bag inlet valve assembly, and the oxygen reservoir can be connected to the same end of the bag.

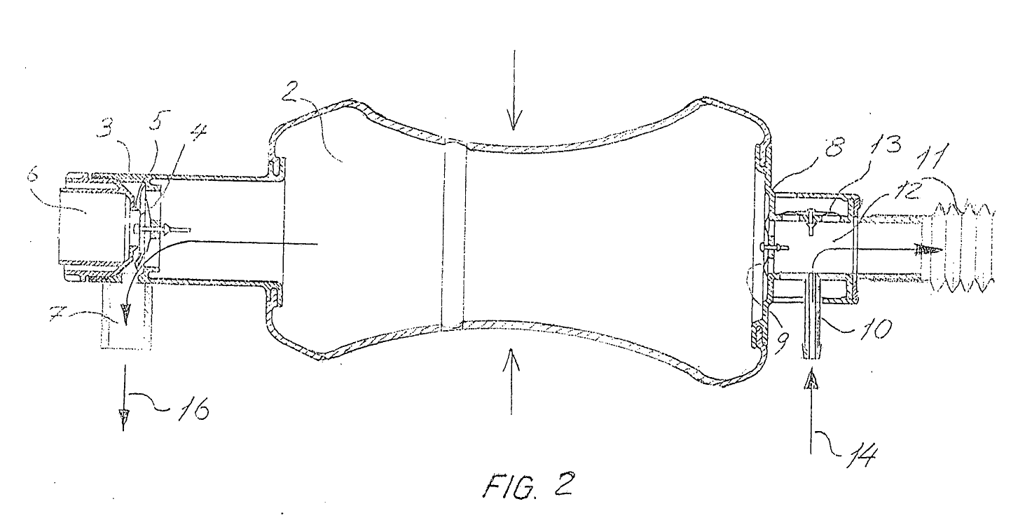

FIG. 2 illustrates the resuscitator during squeezing of the bag 1. A breathing mixture 16 from the interior of the bag 1 flows through the directional control valve 5 to the patient port 7 and an elastomeric control member 4 closes the patient port 7 to the atmosphere 6. Supplemental oxygen 14 continues to push air backwards, out of the oxygen reservoir 11.

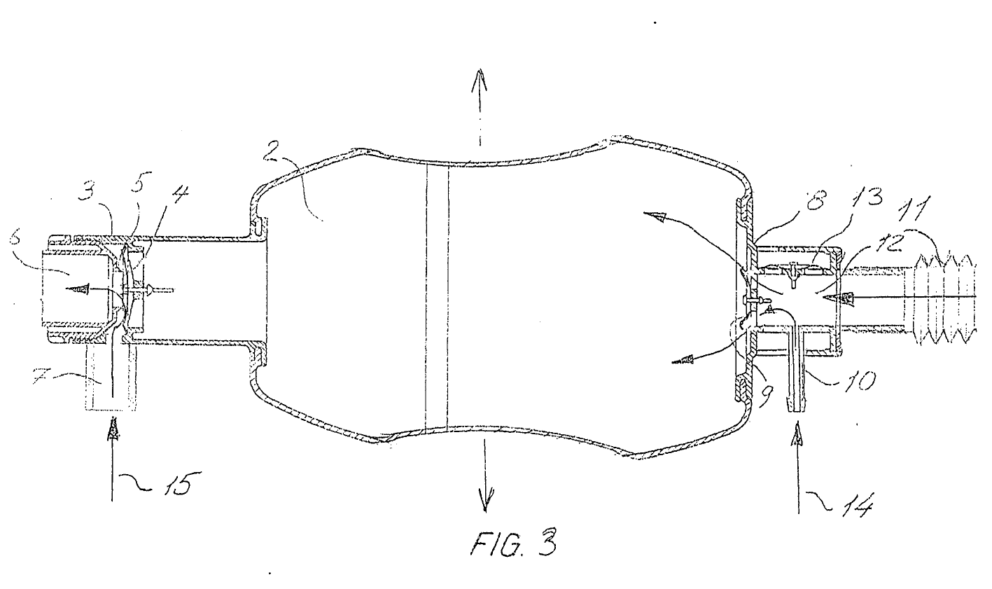

FIG. 3 illustrates the resuscitator during expansion and refilling of the bag 1. The pressure inside the bag 1 is negative and the pressure at the patient port 7 is positive causing the directional control valve 5 to close off the patient port 7 towards the bag 1 and open it for the patient's expiration to flow from patient port 7 to exhaust port 6. The accumulated oxygen in the tubing reservoir 11 is sucked or drawn via a bag inlet valve 9 into the bag 1 together with the inflow of supplementary oxygen 14. The pre-strained pressure relief valve 13 remains closed because the pressure inside the inlet valve conduit 12 is negative.

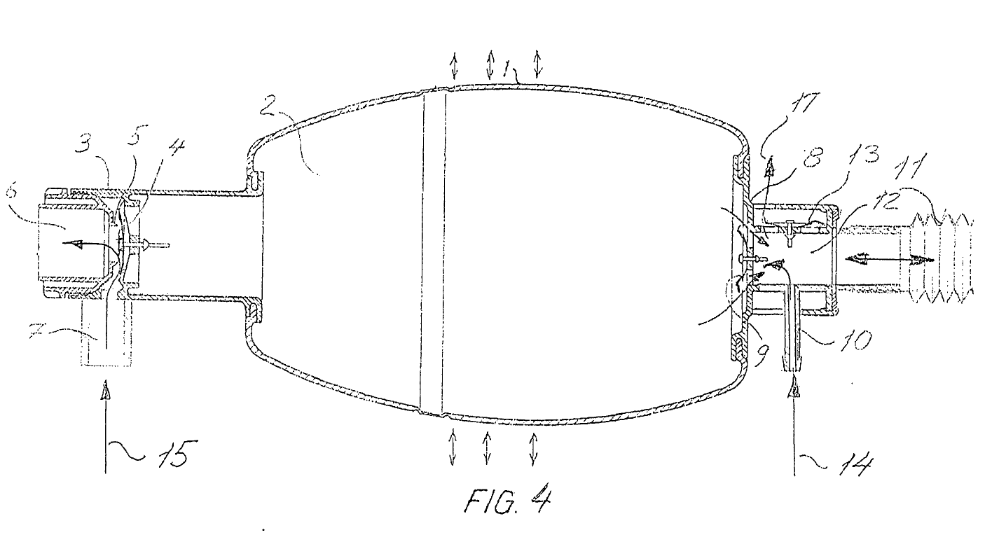

FIG. 4 illustrates the resuscitator in the exact moment when the wall of the bag 1 has unfolded from concave to convex curvature. Expiration is still in progress as indicated by the flow arrow 15 and the bag inlet valve 9 has still not had time to move back and close its valve seat. At this moment, the inertia of the unfolding bag wall material will create deceleration forces, which cause the bag to overshoot the unfolding and deform slightly towards an oval cross section, which in turn creates a reduction of the bag volume and consequently an instantaneous positive pressure wave. This pressure wave is transmitted via the open bag inlet valve 9 to the inlet valve conduit 12 and, in previously known manual resuscitators not having the pre-strained pressure relief valve 13 of the present invention, will tend to start a resonance vibration of the air column in the oxygen reservoir tubing 11. Such resonance pressure fluctuations would in turn be transmitted to the bag and the directional control valve 5 causing it to vibrate between its two positions, thereby allowing a small amount of the expiration mixture to flow into the bag 1 and increase the pressure to a level where the pressure at the patient port 7 is no longer able to open the passage to the exhaust port 6. This will cause the expiration to stop prematurely with a positive pressure still remaining in the lungs.

However, in a resuscitator according to the present invention, the slightly pre-strained one-way relief valve 13 is inserted between the bag inlet valve conduit 12 and the atmosphere in the direction of the arrow 17. The opening pressure, the bias, of the valve is adjusted to relieve the instantaneous pressure wave to the atmosphere in the direction of the arrow 17, thereby limiting the pressure wave energy to a level insufficient to excite a resonance vibration of the air column in the oxygen reservoir tubing 11. As a result, no pressure fluctuations will be transmitted via the bag to the directional control valve 5 which stays open to the atmosphere and allows the expiration from patient port 7 to continue until no positive pressure remains in the lungs of the patient. The opening pressure of valve 13 is at the same time adjusted to a level that will not permit the valve to leak oxygen, when a maximum supplementary flow of at least 15 liters/min is pushing the air column backwards in the oxygen reservoir tubing 11. The opening pressure of valve 13 is preferably adjusted to a bias within the range of 0.3 to 2.0 cm H₂O, dependent on the length of the oxygen reservoir tube and the switching characteristics of the directional control valve 5.

In view of the many changes and modifications that can be made without departing from principles underlying the invention, reference should be made to the appended claims for an understanding of the scope of the protection to be afforded the invention.

What is claimed is:

1. A manual resuscitator comprising a bag, a bag inlet valve assembly and an open oxygen reservoir tube, a conduit between said bag inlet valve assembly and said open oxygen reservoir tube, and a one-way relief valve connecting the conduit to the atmosphere, said one-way relief valve having an opening pressure sufficiently low to prevent excitation of resonance pressure vibrations in said open oxygen reservoir tube.

2. The resuscitator of claim 1 wherein said one-way relief valve comprises an elastomeric valve diaphragm having an outer edge resting against a valve seat and a central portion attached to recessed means of the valve, whereby the elastic deformation of the diaphragm pre-strains the outer edge of the diaphragm against the valve seat.

3. The resuscitator of claim 2 wherein said one-way relief valve has an opening pressure sufficient to substantially prevent leakage of supplementary oxygen through said valve to the atmosphere during backwards flow in said oxygen reservoir tube.

4. The resuscitator of claim 1 wherein said bag has two oppositely disposed ends, a first end having a directional control valve connected thereto and second end having said bag inlet valve assembly and said oxygen reservoir tube attached thereto.

5. The resuscitator of claim 1 having a directional control valve connected to one end of said bag and said bag inlet valve assembly and said oxygen reservoir tube are connected to a same end.

6. A manual resuscitator according to claim 1 having a supply tube for supplementary oxygen connected inside one end of said oxygen reservoir tube and an other end extending through said oxygen reservoir tube and out through an open end.

7. The resuscitator of claim 1 further comprising an inlet for a supply of supplementary oxygen to said conduit connecting said bag inlet valve assembly with said oxygen reservoir tube.
